(19) United States
(12) Patent Application Publication
(10) Pub. No.: US 2025/0345209 A1
(43) Pub. Date: Nov. 13, 2025

(54) DRESSING CAPACITY INDICATOR

(71) Applicant: Solventum Intellectual Properties Company, Maplewood, MN (US)

(72) Inventors: Jonathan G. REHBEIN, San Antonio, TX (US); Javier GONZALEZ, San Antonio, TX (US); Kurt HALVERSON, San Antonio, TX (US)

(21) Appl. No.: 18/868,608

(22) PCT Filed: May 4, 2023
(86) PCT No.: PCT/IB2023/054649

§ 371 Date: Nov. 22, 2024

Related U.S. Application Data
(60) Provisional application No. 63/345,850, filed on May 25, 2022.

(51) Int. Cl. A61F 13/00; A61F 13/0203

(52) U.S. Cl. CPC A61F 13/00055; A61F 13/00987; A61F 13/0203

(57) ABSTRACT

A dressing, system, and method for treating a tissue site is described. The dressing includes a tissue interface having a fluid flow path and an encapsulating film at least partially encapsulating the tissue interface. An indicator is disposed adjacent to the tissue interface proximate to at least one location along the fluid flow path.

### CROSS-REFERENCE TO RELATED APPLICATIONS

This application is a U.S. National Stage Entry of PCT International Application No. PCT/IB2023/054649, filed on May 4, 2023, which claims the benefit of priority to U.S. Provisional Application No. 63/345,850, filed on May 25, 2022, which is incorporated herein by reference in its entirety.

### TECHNICAL FIELD

The invention set forth in the appended claims relates generally to tissue treatment systems and more particularly, but without limitation, to fluid storage containers for use with tissue treatment systems.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for treating tissue in a negative-pressure therapy environment are set forth in the appended claims. Illustrative embodiments are also provided to enable a person skilled in the art to make and use the claimed subject matter.

For example, in some embodiments, a dressing for treating a tissue site is described. The dressing includes a tissue interface having a fluid flow path and an encapsulating film at least partially encapsulating the tissue interface. At least one indicator can be disposed adjacent to the tissue interface proximate to at least one location along the fluid flow path.

More generally, a system for treating a tissue site is described. The system includes a tissue interface having a fluid flow path and a pouch at least partially encapsulating the tissue interface. At least one saturation meter can be disposed adjacent to the tissue interface proximate to at least one location along the fluid flow path. The system can also include a negative-pressure source configured to be fluidly coupled to the tissue interface to draw fluid along the fluid flow path.

In yet other embodiments, a method of manufacturing a dressing for treating a tissue site is described. A tissue interface having a fluid flow path can be provided. The tissue interface can be at least partially encapsulated in a film. At least one indicator can be disposed adjacent to the tissue interface proximate to at least one location along the fluid flow path.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment and instillation treatment in accordance with this specification;

FIG. 2 is an exploded isometric view of an example embodiment of a dressing that can be associated with some embodiments of the therapy system of FIG. 1;

FIG. 3 is a plan view of the dressing of FIG. 2 that can be associated with some embodiments of the therapy system of FIG. 1;

FIG. 4 is a cross-sectional view of the dressing of FIG. 3 taken along line 4-4;

FIG. 5 is a plan view of another example embodiment of a dressing that can be associated with some embodiments of the therapy system of FIG. 1;

FIG. 6 is a cross-sectional view of the dressing of FIG. 5 taken along line 6-6;

FIG. 7 is a top view of an example of an indicator that can be associated with some embodiments of the therapy system of FIG. 1; and

FIG. 8 is front view of the indicator of FIG. 7 illustrating additional details that may be associated with some embodiments.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

The term “tissue site” in the context of the following description broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

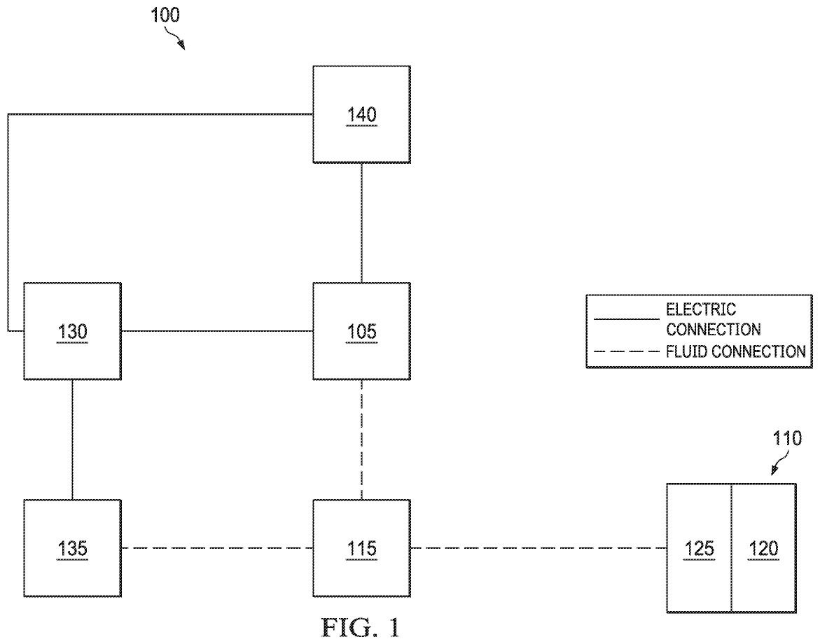

FIG. 1 is a simplified functional block diagram of an example embodiment of a therapy system 100 that can provide negative-pressure therapy with instillation of topical treatment solutions to a tissue site in accordance with this specification. The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a pouch 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy unit.

In general, components of the therapy system 100 may be coupled directly or indirectly. For example, the negative-pressure source 105 may be directly coupled to the pouch 115 and may be indirectly coupled to the dressing 110 through the pouch 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The pouch 115 is representative of a container, canister, or other storage component, which can be used to manage exudates and other fluids withdrawn from a tissue site.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solutions. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ Dressing or V.A.C. VERAFLO™ Dressing, both available from Kinetic Concepts, Inc. of San Antonio, Texas.

The thickness of the tissue interface 120 may also vary according to needs of a prescribed therapy. For example, the thickness of the tissue interface 120 may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in the pouch 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

In some embodiments, the controller 130 may have a continuous pressure mode, in which the negative-pressure source 105 is operated to provide a constant target negative pressure for the duration of treatment or until manually deactivated. Additionally or alternatively, the controller may have an intermittent pressure mode. For example, the controller 130 can operate the negative-pressure source 105 to cycle between a target pressure and atmospheric pressure. For example, the target pressure may be set at a value of 135 mmHg for a specified period of time (e.g., 5 min), followed by a specified period of time (e.g., 2 min) of deactivation. The cycle can be repeated by activating the negative-pressure source 105, which can form a square wave pattern between the target pressure and atmospheric pressure.

In some example embodiments, the increase in negative pressure from ambient pressure to the target pressure may not be instantaneous. For example, the negative-pressure source 105 and the dressing 110 may have an initial rise time. The initial rise time may vary depending on the type of dressing and therapy equipment being used. For example, some therapy systems may increase negative pressure at a rate of about 20-30 mmHg/second, and other therapy systems may increase negative pressure at a rate of about 5-10 mmHg/second. If the therapy system 100 is operating in an intermittent mode, the repeating rise time may be a value substantially equal to the initial rise time.

In some example dynamic pressure control modes, the target pressure can vary with time. For example, the target pressure may vary in the form of a triangular waveform, varying between a negative pressure of 50 and 135 mmHg with a rise rate of negative pressure set at a rate of 25 mmHg/min. and a descent rate set at 25 mmHg/min. In other embodiments of the therapy system 100, the triangular waveform may vary between negative pressure of 25 and 135 mmHg with a rise rate of about 30 mmHg/min. and a descent rate set at about 30 mmHg/min.

In some embodiments, the controller 130 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 130, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

Many dressings may include an absorbent component and are configured to be used with a low-exudating tissue sites. The absorbent component may receive and store liquids, including exudates. In some embodiments, the dressing is in place over the tissue site and the absorbent component receives and stores liquids directly from the tissue site. Preferably, if the absorbent component is at full capacity, the dressing and the absorbent component are removed from the tissue site. The absorbent component may be at full capacity if the absorbent component is saturated. Saturation may be considered a state of the absorbent where no more liquid can be absorbed, leading to free liquid in the dressing. Some clinicians may have difficulty determining if the absorbent component has reached the fluid capacity of the absorbent dressing. Often, a clinician must determine if the absorbent component is at fluid capacity based only on the appearance of the dressing and the absorbent component. In some embodiments, the dressing may be removed before absorbent component reaches fluid capacity, leading to the dressing being removed and replaced too soon. Replacing a dressing prior to the absorbent component reaching fluid capacity increases the frequency of dressing changes that also increase exposure of the tissue site to potential contaminants. Replacing a dressing too soon can also increase the likelihood of damage to tissue surrounding the tissue site through the repeated application of strong adhesives. In other cases, the absorbent component may be beyond fluid capacity when the dressing is removed. If the absorbent component is beyond fluid capacity, excess liquid may remain within the dressing or in contact with tissue. Replacing a dressing too late can cause a build-up of fluid at the tissue site and potentially cause maceration of surrounding tissue.

These issues and others may be addressed by the dressing 110. The dressing 110 may include an absorbent component and an indicator. The indicator may provide a representation of an amount of liquids stored by the absorbent component. By indicating the amount of liquids stored by an absorbent component of the dressing 110, a clinician can more readily determine an appropriate time to remove a dressing having an absorbent component, reducing the risks of tissue site contamination due to too frequent removal of the dressing 110 or maceration due to too infrequent removal of the dressing 110.

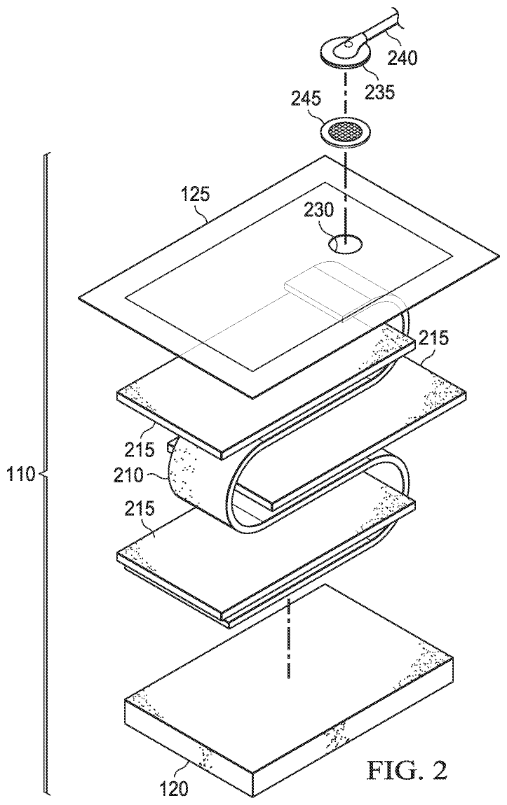

FIG. 2 is an exploded isometric view of an example of the dressing 110 that can be associated with some embodiments of the therapy system 100. As shown in FIG. 2, the dressing 110 may comprise the tissue interface 120, the cover 125, a fluid storage layer, such as an absorbent 210, and one or more fluid indicator layers, such as one or more indicator layers 215, between the absorbent 210. The absorbent 210 and the one or more indicator layers 215 are configured to be sandwiched between the tissue interface 120 and the cover 125.

The cover 125 may include an aperture, such as an outlet 230. In some embodiments, the outlet 230 may include additional components and may form a port. A dressing interface or negative-pressure interface, such as an outlet interface 235, may be placed over the outlet 230 to provide a fluid path between a fluid conductor 240 and an environment over the tissue site provided by the dressing 110. In some embodiments, a filter 245 may be included between the outlet 230 and the outlet interface 235. The filter 245 may be a hydrophobic filter so that fluid communication into the outlet interface 235 and the fluid conductor 240 may be limited to communication of negative pressure, reducing or preventing liquid from flowing into the outlet interface 235 and the fluid conductor 240.

As shown in FIG. 2, the absorbent 210 may have a serpentine shape. The absorbent 210 generally comprises one or more absorbent or absorbent layers, which can provide a means for collecting or storing fluid from the tissue interface 120 to the outlet 230 of the dressing 110 under negative pressure. For example, the absorbent 210 may be adapted to receive negative pressure from a source and distribute negative pressure along the length of the absorbent 210, which may have the effect of collecting fluid from a tissue site and drawing the fluid toward the source.

The absorbent 210 stores, or immobilizes, the liquid from a tissue site. The absorbent 210 may be any substance capable of storing a liquid, such as exudate. For example, the absorbent 210 may form a chemical bond with exudate from the tissue site. Non-limiting examples of the absorbent 210 include super absorbent fiber/particulates, hydrofibre, sodium carboxymethyl cellulose, and/or alginates. In some exemplary embodiments, the absorbent 210 may be formed of a superabsorbent polymer (SAP). Generally, relative to their mass, SAPs can absorb and retain large quantities of liquid, and in particular water. SAPs may be used to hold and stabilize or solidify wound fluids. The SAPs used to form the absorbent 210 may be of the type often referred to as “hydrogels,” “super-absorbents,” or “hydrocolloids.” When disposed within the dressing 110, the SAPs may be formed into fibers or spheres to manifold reduced pressure until the SAPs become saturated. Spaces or voids between the fibers or spheres may allow a reduced pressure that is applied to the dressing 110 to be transferred within and through the absorbent 210. In some embodiments, fibers of the absorbent 210 may be either woven or non-woven. In some embodiments, the absorbent 210 may comprise a substrate in which the SAPs may be dispersed as pellets throughout and/or embedded as a sheet-like layer within the substrate.

The SAPs may be formed in several ways, for example, by gel polymerization, solution polymerization, or suspension polymerization. Gel polymerization may involve blending of acrylic acid, water, cross-linking agents, and ultraviolet (UV) initiator chemicals. The blended mixture may be placed into a reactor where the mixture is exposed to UV light to cause crosslinking reactions that form the SAP. The mixture may be dried and shredded before subsequent packaging and/or distribution. Solution polymerization may involve a water-based monomer solution that produces a mass of reactant polymerized gel. The monomer solution may undergo an exothermic reaction that drives the crosslinking of the monomers. Following the crosslinking process, the reactant polymer gel may be chopped, dried, and ground to its final granule size. Suspension polymerization may involve a water-based reactant suspended in a hydrocarbon-based solvent. However, the suspension polymerization process must be tightly controlled and is not often used.

SAPs absorb liquids by bonding with water molecules through hydrogen bonding. Hydrogen bonding involves the interaction of a polar hydrogen atom with an electronegative atom. As a result, SAPs absorb water based on the ability of the hydrogen atoms in each water molecule to bond with the hydrophilic polymers of the SAP having electronegative ionic components. High absorbing SAPs are formed from ionic crosslinked hydrophilic polymers such as acrylics and acrylamides in the form of salts or free acids. Because the SAPs are ionic, they are affected by the soluble ionic components within the solution being absorbed and will, for example, absorb less saline than pure water. The lower absorption rate of saline is caused by the sodium and chloride ions blocking some of the water absorbing sites on the SAPs. If the fluid being absorbed by the SAP is a solution containing dissolved mineral ions, fewer hydrogen atoms of the water molecules in the solution may be free to bond with the SAP. Thus, the ability of an SAP to absorb and retain a fluid may be dependent upon the ionic concentration of the fluid being absorbed. For example, an SAP may absorb and retain de-ionized water up to 500 times the weight of the dry SAP. In volumetric terms, an SAP may absorb fluid volumes as high as 30 to 60 times the dry volume of the SAP. Other fluids having a higher ionic concentration may be absorbed at lower quantities. For example, an SAP may only absorb and retain a solution that is 0.9% salt (NaCl) up to 50 times the weight of the dry SAP. Since wound fluids contain salts, such as sodium, potassium, and calcium, the absorption capacity of the SAP may be reduced if compared to the absorption capacity of deionized water.

In some embodiments, the absorbent 210 may comprise a KERRAMAX CARE™ Super-Absorbent Dressing material available from Kinetic Concepts, Inc. of San Antonio, Texas. For example, the absorbent 210 may comprise a superabsorbent laminate comprised of 304 g.s.m. FAVOR-PAC™ 230 superabsorbent powder glued by PAFRA™ 8667 adhesive between two layers of 50 g.s.m. LIDRO™ non-woven material. In some embodiments, the absorbent 210 may comprise an absorbent available from Gelok International. The presence of the absorbent 210 may also help to minimize fluid loss or reflux.

Each of the indicator layers 215 may be a layer of a film material having an ultra-low index coating. For example, the indicator layer 215 can be a porous polymeric film having a network of a plurality of interconnected voids dispersed in a polymeric binder or matrix material along with other particles. In some embodiments at least some of the voids can be connected to each other via hollow tunnels or hollow tunnel-like passages. The indicator layer 215 can be optically diffusive to at least one wavelength of light when the network of interconnected voids is substantially free of fluid. The indicator layer 215 can undergo detectable optical change upon liquid ingress into the network of interconnected voids or liquid egress from the network of interconnected voids.

Each indicator layer 215 may include a first polymeric film on a first major surface of the porous polymeric film and a second polymeric film on a second major surface of the porous polymeric film. The second polymeric film may be different from the first polymeric film. The first polymeric film may be transmissive to visible light, and the second polymeric film includes at least one of a pigment, a dye, an indicia, and combinations thereof.

Some porous materials support total internal reflection (TIR) or enhanced internal reflection (EIR) by virtue of including a plurality of voids. When light that travels in an optically clear non-porous medium is incident on a stratum possessing high porosity, the reflectivity of the incident light is much higher at oblique angles than at normal incidence. In the case of no or low haze voided films, the reflectivity at oblique angles greater than the critical angle is close to about 100%. In such cases, the incident light undergoes total internal reflection (TIR). In the case of high haze voided porous materials, the oblique angle reflectivity can be close to 100% over a similar range of incident angles even though the light may not undergo TIR. This enhanced reflectivity for high haze films is similar to TIR and is designated as Enhanced Internal Reflectivity (EIR). As used herein, by a porous or voided material having enhanced internal reflection (EIR), the reflectance at the boundary of the voided and non-voided strata of the layer is greater with the voids than without the voids.

In some embodiments, the voids in the porous materials have an index of refraction nᵥ and a permittivity εᵥ. where nᵥ²=εᵥ and the binder has an index of refraction no and a permittivity ε_{b}, where n_{b}²=ε_{b}. In general, the interaction of a porous material with light, such as light that is incident on, or propagates in, a layer of the material, depends on a number of film characteristics such as, for example, the layer thickness, the binder index, the void or void index, the void shape and size, the spatial distribution of the voids, and the wavelength of light. In some embodiments, light that is incident on or propagates within the gradient porous material, “sees” or “experiences” an effective permittivity ε_{eff} and an effective index n_{eff}, where n_{eff} can be expressed in terms of the void index nᵥ the binder index n_{b}, and the void porosity or volume fraction “f” In such cases, the porous materials are sufficiently thick, and the voids are sufficiently small, so that light cannot resolve the shape and features of a single or isolated void. In such cases, the size of at least a majority of the voids, such as at least 60% or 70% or 80% or 90% of the voids, is not greater than about λ/5, or not greater than about λ/6, or not greater than about λ/8, or not greater than about λ/10, or not greater than about λ/20, where λ is the wavelength of light.

In some embodiments, light that is incident on a porous material is a visible light meaning that the wavelength of the light is in the visible range of the electromagnetic spectrum. In such cases, the visible light has a wavelength that is in a range from about 380 nm to about 750 nm, or from about 400 nm to about 700 nm, or from about 420 nm to about 680 nm. In such cases, the porous material has an effective index of refraction and includes a plurality of voids if the size of at least a majority of the voids, such as at least 60% or 70% or 80% or 90% of the voids, is not greater than about 70 nm, or not greater than about 60 nm, or not greater than about 50 nm, or not greater than about 40 nm, or not greater than about 30 nm, or not greater than about 20 nm, or not greater than about 10 nm.

In some embodiments, the porous materials are sufficiently thick so that a layer of the material can reasonably have an effective index that can be expressed in terms of the indices of refraction of the voids and the binder, and the void or void volume fraction or porosity. In such cases, the thickness of the layer of the porous material is not less than about 100 nm, or not less than about 200 nm, or not less than about 500 nm, or not less than about 700 nm, or not less than about 1,000 nm.

If the voids in the porous material are sufficiently small and the optical film is sufficiently thick, a layer of the material has an effective permittivity ε_{eff} that can be expressed as:

$${ε}_{eff} = f {ε}_{v} + ( 1 - f ) {ε}_{eff} \qquad ( 1 )$$

The effective index n_{eff} of the porous layer can be expressed as:

$${n}_{eff}^{2} = {fn}_{v}^{2} + ( 1 - f ) {n}_{b}^{2} \qquad ( 2 )$$

In some embodiments, such as when the difference between the indices of refraction of the voids and the binder is sufficiently small, the effective index of the layer of the porous material can be approximated by the following expression:

$${n}_{eff} = {fn}_{v} + ( 1 - f ) {n}_{b} \qquad ( 3 )$$

The effective refractive index of the layer of the porous material is the volume weighted average of the indices of refraction of the voids and the binder. For example, a porous material that has a void volume fraction of about 50% and a binder that has an index of refraction of about 1.5, has an effective index of about 1.25. In some embodiments, the preferred refractive index of the at least one indicator 212 is less than 1.35.

The voids can have a size d₁ that can generally be controlled by choosing suitable composition and fabrication techniques, such as coating, drying and curing conditions. In general, d₁ can be any desired value in any desired range of values. For example, in some embodiments, at least a majority of the voids, such as at least 60% or 70% or 80% or 90% or 95% of the voids, have a size that is in a desired range. For example, in some embodiments, at least a majority of the voids, such as at least 60% or 70% or 80% or 90% or 95% of the voids, have a size that is not greater than about 10 microns, or not greater than about 7 microns, or not greater than about 5 microns, or not greater than about 4 microns, or not greater than about 3 microns, or not greater than about 2 microns, or not greater than about 1 micron, or not greater than about 0.7 microns, or not greater than about 0.5 microns.

In some embodiments, the plurality of interconnected voids can have an average void or void size that is not greater than about 5 microns, or not greater than about 4 microns, or not greater than about 3 microns, or not greater than about 2 microns, or not greater than about 1 micron, or not greater than about 0.7 microns, or not greater than about 0.5 microns.

In some embodiments, some of the voids can be sufficiently small so that their primary optical effect is to reduce the effective index, while some other voids can reduce the effective index and scatter light, while still some other voids can be sufficiently large so that their primary optical effect is to scatter light.

In some embodiments, the primary optical effect of the voids is to affect the effective index of layer of the porous material. For example, in such cases, d₁ are not greater than about λ/5, or not greater than about λ/6, or not greater than about λ/8, or not greater than about λ/10, or not greater than about λ/20, where λ is the wavelength of light. As another example, in such cases, d₁ is not greater than about 70 nm, or not greater than about 60 nm, or not greater than about 50 nm, or not greater than about 40 nm, or not greater than about 30 nm, or not greater than about 20 nm, or not greater than about 10 nm. In such cases, the voids may also scatter light, but the primary optical effect of the voids is to define an effective medium that has an effective index. The effective index depends, in part, on the indices of refraction of the voids, the binder, and any other particles that may comprise the porous material. In some embodiments, the effective index is a reduced effective index, meaning that the effective index is less than the index of the binder and the index of the particles.

In embodiments where the primary optical effect of the voids is to affect the index, d₁ is sufficiently small so that a substantial fraction, such as at least about 60%, or at least about 70%, or at least about 80%, or at least about 90%, or at least about 95% of the voids have the primary optical effect of reducing the effective index. In such cases, a substantial fraction, such as at least about 60%, or at least about 70%, or at least about 80%, or at least about 90%, or at least about 95% of the voids have a size that is in a range from about 1 nm to about 200 nm, or from about 1 nm to about 150 nm, or from about 1 nm to about 100 nm, or from about 1 nm to about 50 nm, or from about 1 nm to about 20 nm.

In some embodiments, the index of refraction nᵢ of the particles can be sufficiently close to the index n_{b} of the binder, so that the effective index does not depend, or depends very little, on the index of refraction of the particles. In such cases, the difference between nᵢ and n_{b} is not greater than about 0.01, or not greater than about 0.007, or not greater than about 0.005, or not greater than about 0.003, or not greater than about 0.002, or not greater than about 0.001.

In embodiments where the primary optical effect of network of voids is to affect the effective index and not to, for example, scatter light, the optical haze of the layer of porous material that is due to the voids is not greater than about 5%, or not greater than about 4%, or not greater than about 3.5%, or not greater than about 4%, or not greater than about 3%, or not greater than about 2.5%, or not greater than about 2%, or not greater than about 1.5%, or not greater than about 1%. In such cases, the effective index of the effective medium of the layer of porous material is not greater than about 1.35, or not greater than about 1.3, or not greater than about 1.25, or not greater than about 1.2, or not greater than about 1.15, or not greater than about 1.1, or not greater than about 1.05.

In embodiments where the layer of porous material can reasonably have a reduced effective index, the thickness of the layer is not less than about 100 nm, or not less than about 200 nm, or not less than about 500 nm, or not less than about 700 nm, or not less than about 1,000 nm, or not less than about 1500 nm, or not less than about 2000 nm.

In some embodiments, d₁ is sufficiently large so that the primary optical effect is to scatter light and produce optical haze. In such cases, d₁ is not less than about 200 nm, or not less than about 300 nm, or not less than about 400 nm, or not less than about 500 nm, or not less than about 600 nm, or not less than about 700 nm, or not less than about 800 nm, or not less than about 900 nm, or not less than about 1000 nm. In such cases, the voids may also affect the index, but their primarily optical effect is to scatter light. In such cases, light incident on the layer can be scattered by both the voids and the particles.

In some embodiments, the layer of porous material has a low optical haze. In such cases, the optical haze of the layer is not greater than about 5%, not greater than about 4%, not greater than about 3.5%, not greater than about 4%, not greater than about 3%, not greater than about 2.5%, not greater than about 2%, not greater than about 1.5%, or not greater than about 1%. In such cases, the layer of porous material can have a reduced effective index that is not greater than about 1.35, not greater than about 1.3, not greater than about 1.2, not greater than about 1.25, not greater than about 1.1, or not greater than about 1.05. For light nominally incident on the layer of the porous material, optical haze, as used herein, is defined as the ratio of the transmitted light that deviates from the normal direction by more than 4 degrees to the total transmitted light. Haze values were measured using a HAZE-GARD PLUS haze meter (available from BYK-Gardner, Silver Springs, Md.) according to the procedure described in ASTM D 1003.

In some embodiments, the layer of the porous material has a high optical haze. In such cases, the haze of the porous material is not less than about 40%, not less than about 50%, not less than about 60%, not less than about 70%, not less than about 80%, not less than about 90%, or not less than about 95%. In some embodiments, the layer can have an intermediate optical haze, for example, between about 5% and about 50% optical haze.

In some embodiments, the layer of the porous material has a high diffuse optical reflectance. In such cases, the diffuse optical reflectance of the layer is not less than about 30%, not less than about 40%, not less than about 50%, or not less than about 60%.

In some embodiments, the layer of the porous material has a high optical clarity. For light nominally incident on the layer, optical clarity, as used herein, refers to the ratio (T2−T1)/(T1+T2), where T1 is the transmitted light that deviates from the nominal direction between 1.6 and 2 degrees, and T2 is the transmitted light that lies between zero and 0.7 degrees from the normal direction. Clarity values were measured using a Haze-Gard Plus haze meter from BYK-Gardner. In the cases where the layer of the porous material 300A has a high optical clarity, the clarity is not less than about 40%, or not less than about 50%, or not less than about 60%, or not less than about 70%, or not less than about 80%, or not less than about 90%, or not less than about 95%. In some embodiments, the layer of the porous material, i.e., the ultra-low index coating, can have a transparency between about 50% and about 90% or can increase in transparency in response to exposure to liquid by greater than 25%.

In some embodiments, the layer of the porous material has a low optical clarity. In such cases, the optical clarity of the layer is not greater than about 10%, not greater than about 7%, not greater than about 5%, not greater than about 4%, not greater than about 3%, not greater than about 2%, or not greater than about 1%.

In general, the layer of the porous material can have any porosity or void volume fraction that may be desirable in an application. In some embodiments, the volume fraction of plurality of voids in the layer is not less than about 20%, not less than about 30%, not less than about 40%, not less than about 50%, not less than about 60%, not less than about 70%, not less than about 80%, or not less than about 90%.

In some embodiments, the layer of the porous material can manifest some low-index properties, even if the film has a high optical haze and/or diffuse reflectance. For example, in such cases, the layer can support TIR at angles that correspond to an index that is smaller than the index no of the binder.

The binder can be or can include any material that may be desirable in an application. For example, in some embodiments, which are not intended to be limiting, the binder can be derived from thermosetting, thermoplastic, and UV curable polymeric materials. Examples include, but are not limited to, polyvinyl alcohol (PVA), polyvinyl butyral (PVB), polyvinyl pyrrolidone (PVP),5 polyethylene vinyl acetate copolymers (EVA), cellulose acetate butyrate (CAB), polyurethanes (PURs), polymethylmethacrylate (PMMA), polyacrylates, epoxies, silicones, and fluoropolymers. The binders can be soluble in a suitable solvent such as, for example, water, ethyl acetate, acetone, 2-butone, and the like, and can be used as dispersions or emulsions.

Examples of some commercially available binders useful in the mixtures are those available 10 from Kuraray-USA, Wacker Chemical, Dyncon LLC, and Rohm and Haas. Although the binder can be a polymeric system, it can also be added as a polymerizable monomeric system, such as a UV, or thermally curable or crosslinkable system. Examples of such systems would be UV polymerizable acrylates, methacrylates, multi-functional acrylates, urethane-acrylates, and mixtures thereof. Some typical examples would be 1,6 hexane diol diacrylate, trimethylol propane triacrylate, pentaerythritol15 triacryalate. Such systems are readily available from suppliers such as Neo Res (Newark, DE), Arkema (Philadelphia, PA), or Sartomer (Exton, PA). Actinic radiation such as electron beam (Ebeam), gamma, and UV radiation are useful methods to initiate the polymerization of these systems, with many embodiments utilizing UV active systems. Other useful binder systems can also be cationically polymerized, such systems are available as vinyl ethers and epoxides.

The polymeric binders can also be formulated with cross linkers that can chemically bond with the polymeric binder to form a crosslinked network. Although the formation of crosslinks is not a prerequisite for the formation of the porous structure or the low refractive index optical properties, itis often desirable for other functional reasons such as to improve the cohesive strength of the coating, adhesion to the substrate or moisture, or thermal and solvent resistance. The specific type of crosslinker is dependent upon the binder used. Typical crosslinkers for polymeric binders such as PVA would be di-isocyanates, titantates such as those available under the trade designation TYZORLAf from DowDuPont, Midland, MI, poly (epichlorhydrin) amide adducts such as PolyCup 172, (available from Hercules, Wilmington, DE), multi-functional aziridines such as CX100 (available from Neo-Res, Newark, DE) and boric acid, di-epoxides, diacids and the like.

The polymeric binders may form a separate phase with the particle aggregates or may be inter-dispersed between the particle aggregates in a manner to “bind” the aggregates together into a strictures that connect with the metal oxidize particles through direct covalent bond formation or molecular interactions such as ionic, dipole, van Der Waals forces, hydrogen bonding and physical entanglements with the metal oxides.

Optical film can be produced using methods that may be desirable in an application. Generally, in one process, first a solution is prepared that includes a plurality of particles, such as nanoparticles, and a polymerizable material dissolved in a solvent, where the polymerizable material can include, for example, one or more types of monomers. Next, the polymerizable material is polymerized, for example by applying heat or light, to form an insoluble polymer matrix in the solvent. In one example, the polymerization occurs in an environment that has an elevated level of oxygen adjacent one of the surfaces, inhibiting the polymerization near that surface to create a gradient optical film. In one example, a concentration of photo initiator near one of the surfaces is increased relative to another surface, to create a gradient optical film.

In some embodiments, after the polymerization step, the solvent may still include some of the polymerizable material, although at a lower concentration. Next, the solvent is removed by drying or 10 evaporating the solution resulting in optical film that includes a network, or a plurality, of voids dispersed in polymer binder. Optical film further includes plurality of particles dispersed in the polymer. Particles are bound to binder, where the bonding can be physical or chemical, or be encapsulated by binder.

The porous material can have other materials in addition to binders and particles. For example, porous polymeric film can include one or more additives, such as for example, coupling agents, to help wet the surface of a substrate, on which the layer is formed. As another example, the porous layer can include one or more colorants, such a carbon black, for imparting a color, such as the black color, to the layer. Other exemplary materials in the layer of the porous material include initiators, such as one or more photo-initiators, anti-stats, UV absorbers and release agents. In some embodiments, the layer can include adown converting material that is capable of absorbing light and reemitting a longer wavelength light. Exemplary down-converting materials include phosphors.

In general, a porous layer can have a desirable porosity for any weight ratio of binder to plurality of particles. Accordingly, in general, the weight ratio can be any value that may be desirable in an application. In some embodiments, the weight ratio of binder to plurality of particles is not less than about 1:2.5, not less than about 1:2.3, not less than about 1:2, not less than about 1:1, not less than about 1.5:1, not less than about 2:1, not less than about 2.5:1, not less than about 3:1, not less than about 3.5:1, not less than about 4:1, or not less than about 5:1. In some embodiments, the weight ratio is in a range from about 1:2.3 to about 4:1.

In some embodiments, top major surface of the layer of the porous material can be treated to, for example, improve the adhesion of optical film to another layer. For example, top major surface can be corona treated.

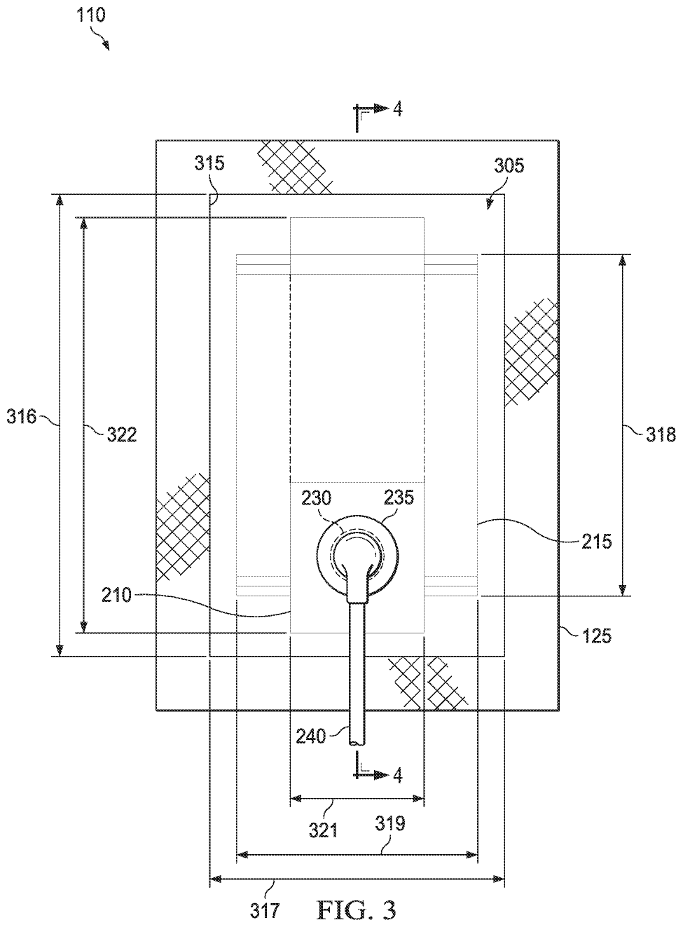

FIG. 3 is a plan view of the dressing 110 of FIG. 2. The cover 125 may be coupled on peripheral portions of the cover 125 to tissue adjacent the tissue site to form an interior space 305 containing the absorbent 210, the one or more indicator layers 215, and the tissue interface 120. The cover 125 may be adhered to tissue surrounding a tissue site. Suitable bonds between the cover 125 and tissue surrounding a tissue site may include pressure-sensitive adhesive. In some embodiments, the adhesion between the cover 125 and the tissue surrounding the tissue site may form an inner boundary line 315 of the interior space 305.

The dressing 110 may have a length 316 and a width 317 along the inner boundary line 315. In some embodiments, the length 316 may be in a range of about 5 centimeters to about 20 centimeters and the width 317 may be in a range of about 5 centimeters to about 20 centimeters. In some embodiments, the length 316 may be about 11 centimeters and the width 317 may be about 8.5 centimeters. The length 316 and the width 317 along the inner boundary line 315 may define a first planar area A1. In some embodiments, the first planar area A; may be in a range of about 50 cm² to about 150 cm². In some embodiments, the first planar area A; may be in a range of about 75 cm² to about 100 cm². In some embodiments, the first planar area A; may be about 93.5 cm². The dimensions of the dressing 110 may vary according to a prescribed therapy or application. With the expansion properties of the absorbent 210 during liquid fluid absorption, the dimensions of the dressing 110 may determine the liquid volume capacity of the dressing 110.

The indicator layers 215 may have a length 318 and a width 319. In some embodiments, the length 318 of the indicator layers 215 may be less than or equal to the length 316 of the dressing 110. In some embodiments, the length 318 of the indicator layers 215 may be equal to the length 316 of the dressing 110 minus a thickness of the indicator layers 215. In some embodiments, the width 319 of the indicator layers 215 may be less than or equal to the width 317 of the dressing 110. In some embodiments, the width 319 of the indicator layers 215 may be about equal to the width 317 of the dressing 110. In some embodiments, the length 318 may be in a range of about 5 centimeters to about 20 centimeters, and the width 319 may be in a range of about 5 centimeters to about 20 centimeters. In some embodiments, the length 318 may be about 9 centimeters, and the width 319 may be about 7 centimeters. In some embodiments, the length 318 may be about 8.5 centimeters, and the width 319 may be about 8.5 centimeters. The indicator layers 215 may have a second planar area A₂. In some embodiments, the second planar area A₂ may be less than or equal to the first planar area A1. In some embodiments, the second planar area A₂ may be in a range of about 50 cm² to about 150 cm². In some embodiments, the second planar area A₂ may be in a range of about 50 cm² to about 100 cm². In some embodiments, the second planar area A₂ may be about 63 cm². The dimensions of the indicator layers 215 may vary according to a prescribed therapy or application.

As further shown in the example of FIG. 3, the absorbent 210 may have a length 322 and a width 321. In some embodiments, the width 321 of the absorbent 210 may be less than the width 319 of the one or more indicator layers 215. The narrower width 321 of the absorbent 210 in comparison to the width 319 of the one or more indicator layers 215 may focus the flow of fluid along the serpentine fluid path of the absorbent 210 and may reduce bleed over around the sides of the absorbent 210. In some embodiments, the narrower width 321 of the absorbent 210 may permit the indicator layers 215 to be visible through the cover 125 if the absorbent 210 is saturated. In some embodiments, the length of the absorbent 210 can be equal to the length 316 of the dressing 110 minus a thickness of the absorbent 210.

In some embodiments, the indicator layers 215 may comprise one or more baffles, barriers, or weirs, such as baffles disposed in the dressing 110. In some embodiments, the baffles may be formed from or include a polymer film. For example, a surface of the indicator layers 215 facing the tissue site may comprise a polymer film. In some embodiments, the baffles may comprise a thermoplastic film or sheet. The baffles may comprise, for example, one or more of the following materials: thermoplastic polyurethane (TPU); polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. In some embodiments, the baffles may comprise one or more of a polymer film and a backing layer, such as, for example, a casting paper, a film, or polyethylene. Further, in some embodiments, the backing layer may be a polyester material such as polyethylene terephthalate (PET), or similar polar semi-crystalline polymer. In some embodiments, the baffles may be fluid impermeable. For example, the baffles may be configured to prevent the passage of liquid and gas through the baffles. In some embodiments, the baffles may be configured to prevent the passage of liquid, but allow the passage of gas or vapor, through the baffles. In some embodiments, the baffles may be formed of a material that is liquid impermeable. In some embodiments, the baffles may be formed of a material that is gas impermeable. In some embodiments, the baffles may be formed of a material that is liquid impermeable but gas permeable.

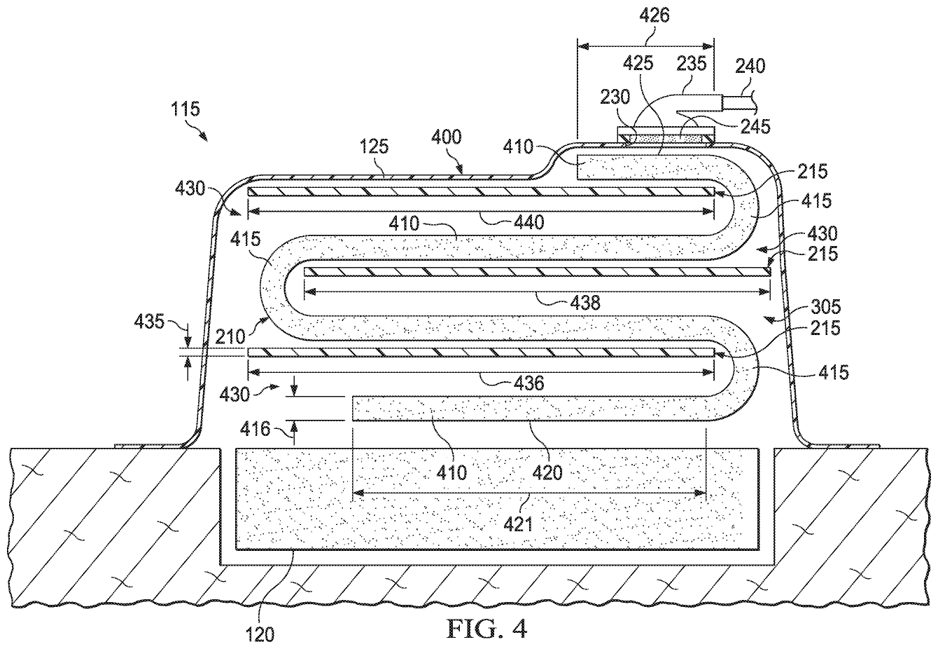

FIG. 4 is a cross-sectional view of the dressing 110 of FIG. 3 taken along line 4-4. As shown in FIG. 4, the absorbent 210 and the one or more indicator layers 215 may be disposed in the interior space 305 of the dressing 110. The outlet 230 may be fluidly coupled with the interior space 305.

In some embodiments, the absorbent 210 may having a serpentine shape comprising a plurality of layers 410 and a plurality of connectors 415. For example, the absorbent 210 may comprise a population Nr. of layers 410 and a population N_{C} of connectors 415, wherein the layers 410 and the connectors 415 are fluidly coupled. In some embodiments, each of the layers 410 of the absorbent 210 may be parallel to one another. In some embodiments, the connectors 415 may be curved. In some embodiments, the population N_{C} of connectors 415 may be one less than the population Ny. of layers 410 (e.g., N_{C}=N_{L}−1). The layers 410 and connectors 415 of the absorbent 210 may have a thickness 416 in a range of about 1 millimeter to about 5 millimeters. In some embodiments, the thickness 416 may be about 2.5 millimeters.

The layer 410 of the absorbent 210 proximate to the tissue interface 120 may be considered a first layer, such as an inlet layer 420. The layer 410 of the absorbent 210 proximate to the outlet 230 may be considered a second layer, such as an outlet layer 425. The inlet layer 420 may have a first length 421 and the outlet layer 425 may have a second length 426. In some embodiments, the second length 426 may be less than the first length 421. In some embodiments, the second length 426 may be equal to the first length 421. As further shown in FIG. 4, the inlet layer 420 of the absorbent 210 may cover at least a portion of the tissue interface 120, and the outlet layer 425 of the absorbent 210 may cover the outlet 230. In some embodiments, the inlet layer 420 may be coextensive with a surface of the tissue interface 120. In other embodiments, the inlet layer 420 may have an area less than an area of the surface of the tissue interface 120 so that the inlet layer 420 and the surface of the tissue interface 120 are not coextensive. The outlet layer 425 may extend beyond the outlet 230.

In some embodiments, a portion of the absorbent 210, such as the inlet layer 420, may be between the tissue interface 120 and an indicator layer 215. In some embodiments, a portion of the absorbent 210, such as the outlet layer 425, may be between the outlet 230 and an indicator layer 215.

A population N_{G} of gaps 430 may be located between the layers 410. In some embodiments, the population N_{G} of gaps 430 may be one less than the population Ny. of layers 410 (e.g., N_{G}=N_{L}−1). In some embodiments, the population N_{G} of gaps 430 may be equal to the population N_{C} of connectors 415 (e.g., N_{G}=N_{C}).

The dressing 110 may comprise a population N_{A} of indicator layers 215. In some embodiments, an indicator layer 215 may be disposed in each gap 430. The indicator layers 215 may be proximate the layers 410 of the absorbent 210. In some embodiments, the population N_{A} of the indicator layers 215 may be one less than the population Ny. of layers 410 (e.g., N_{A}=N_{L}−1). In some embodiments, the population N_{A} of the indicator layers 215 may be equal to the population N_{C} of connectors 415 (e.g., N_{A}=N_{C}). In some embodiments, the population N_{A} of the indicator layers 215 may be equal to the population N_{G} of gaps 430 (e.g., N_{A}=N_{G}). The indicator layers 215 may have a thickness 435 in a range of about 0.05 millimeters to about 5 millimeters when dry. In some embodiments, the thickness 435 may be about 1 millimeter when dry.

As shown in the example of FIG. 4, the population Ny. of layers 410 may be 4, the population N_{C} of connectors 415 may be 3, the population N_{G} of gaps 430 may be 3, and the population N_{A} of indicator layers 215 may be 3. Each of the indicator layers 215 can be disposed in a respective gap 430. Each of the indicator layers 215 may protrude from the gap 430 beyond the overlying connector 415. Similarly, each of the indicator layers 215 may protrude beyond the indicator layer 215 overlying it. Thus, the indicator layer 215 proximate the tissue site may be visible from an exterior of the cover 125. The indicator layer 215 disposed in an intermediate position between the tissue site and the cover 125 may be visible from an exterior of the cover 125, and the indicator layer 215 disposed between the absorbent 210 and the cover 125 is visible form an exterior of the cover 125. In some embodiments, the indicator layer 215 proximate the tissue interface 120 may have a length 436, the indicator layer 215 disposed in the intermediate position between the tissue interface 120 and the cover 125 may have a length 438, and the indicator layer 215 disposed between the absorbent 210 and the cover 125 may have a length 440. The length 436 may be greater than the length 438 and the length 440, and the length 438 may be greater than the length 440.

In operation, the tissue interface 120 can be positioned adjacent the tissue site, and the dressing 110 applied over the tissue interface 120. The dressing 110 can be fluidly coupled to a source of negative pressure, such as the negative-pressure source 105, and the negative-pressure source 105 can be operated to draw fluid from the interior space 305. In some embodiments, the indicator layers 215, acting as baffles, can direct fluid flow through a tortuous fluid path through the absorbent 210. As fluid is drawn from the interior space 305, liquids at the tissue site can be drawn through the tissue interface 120 and into the absorbent 210. For example, liquids can be drawn into the inlet layer 420. As liquids are drawn into the inlet layer 420, the inlet layer 420 may absorb the liquids. Continued negative-pressure therapy may cause the inlet layer 420 to become saturated. Saturation of the inlet layer 420 can cause free liquid to interact with the indicator layer 215 proximate to the inlet layer 420 as the free liquid is drawn into the layer 410 immediately overlying the inlet layer 420. As the liquid interacts with the indicator layer 215, the liquid can cause the indicator layer 215 to transition. For example, the indicator layer 215 may transition from opaque to clear. In some embodiments, the indicator layer 215 may permit a user to visually see the inlet layer 420 following the transition. In other embodiments, the indicator layer 215 may transition from a first color to a second color. For example, the indicator layer 215 may transition from opaque, hazy, or white to a contrasting color. For example, the indicator layer 215 may become yellow, green, blue, or a fluorescent version of a desired color. The user may see the color through the cover 125, indicating to the user that the inlet layer 420 may be saturated.

As more fluid is drawn into the absorbent 210, subsequent indicator layers 215 disposed between the tissue interface 120 and the cover 125 may subsequently transition as the layer 410 of the absorbent 210 becomes saturated. If all indicator layers 215 have transitioned, the absorbent 210 is saturated and the dressing 110 can be removed and changed.

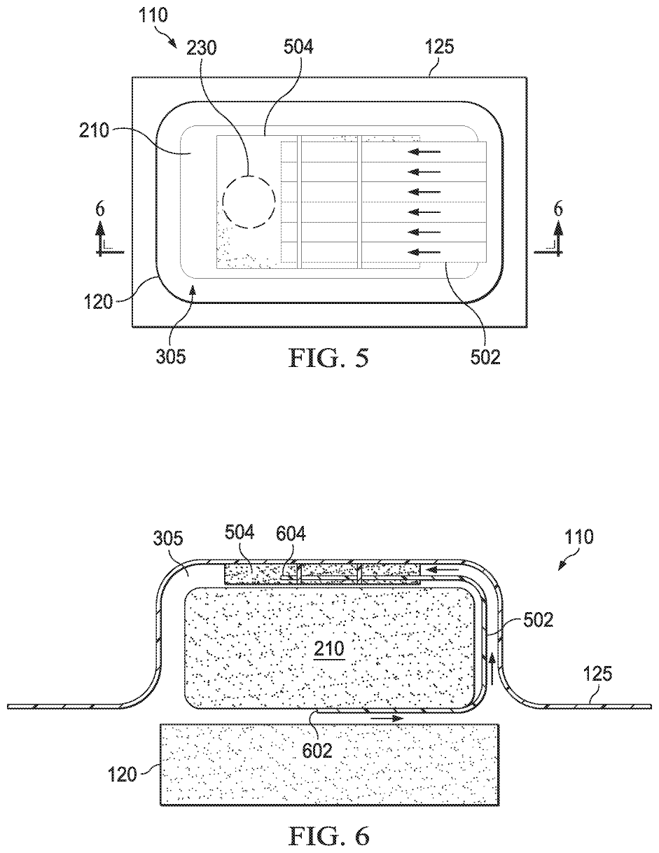

FIG. 5 is a plan view of another dressing 110 that may be associated with some embodiments of the system 100 of FIG. 1. The dressing 110 may include the tissue interface 120 configured to be disposed at the tissue site and the cover 125 disposed over the tissue interface 120 and the tissue site to create the interior space 305. The absorbent 210 can be disposed in the interior space 305. In some embodiments, the absorbent 210 can be positioned adjacent to the tissue interface 120. In some embodiments, the absorbent 210 can be positioned between the tissue interface 120 and the cover 125. The cover 125 may include the outlet 230 for fluid coupling of the dressing 110 to the negative-pressure source 105.

The dressing 110 can also include a plurality of fluid conductors, microfluidic channels, or a fluid control film 502 and an indicator or a timer 504. The timer 504 can be an indicator having an ultra-low index coating similar to those referenced above with respect to the indicator layers 215.

The fluid control film 502 can comprise sheets or films having micro structured surfaces including a plurality of open channels having a high aspect ratio (that is, channel length divided by the wetted channel perimeter), rather than a mass of fibers. The channels of fluid control films preferably provide more effective liquid flow than is achieved with webs, foam, or those formed from fibers. The channels in the present invention are precisely replicated, with high fidelity, from a predetermined pattern and form a series of individual open capillary channels that extend along a major surface. These micro-replicated channels formed in sheets, films, or tubes are preferably uniform and regular along substantially each channel length and more preferably from channel to channel. Fluid control films can be formed from any thermoplastic material suitable for casting, or embossing including, for example, polyolefins, polyesters, polyamides, poly (vinyl chloride), polyether esters, polyimides, polyesteramide, polyacrylates, polyvinylacetate, hydrolyzed derivatives of polyvinylacetate, etc. Polyolefins are preferred, particularly polyethylene or polypropylene, blends and/or copolymers thereof, and copolymers of propylene and/or ethylene with minor proportions of other monomers, such as vinyl acetate or acrylates such as methyl and butylacrylate. Polyolefins are preferred because of their excellent physical properties, case of processing, and typically lower cost than other thermoplastic materials having similar characteristics. Polyolefins readily replicate the surface of a casting or embossing roll. They are tough, durable and hold their shape well, thus making such films easy to handle after the casting or embossing process. Hydrophilic polyurethanes are also preferred for their physical properties and inherently high surface energy.

Alternatively, fluid control films can be cast from thermosets (curable resin materials) such as polyurethanes, acrylates, epoxies and silicones, and cured by exposure to heat or UV or E-beam radiation, or moisture. These materials may contain various additives including surface energy modifiers (such as surfactants and hydrophilic polymers), plasticizers, antioxidants, pigments, release agents, antistatic agents, and the like. Suitable fluid control films also can be manufactured using pressure sensitive adhesive materials. In some embodiments, the channels may be formed using inorganic materials (e.g., glass, ceramics, or metals). Preferably, the fluid control film substantially retains its geometry and surface characteristics upon exposure to liquids. The fluid control film may also be treated to render the film biocompatible. For example, a heparin coating may be applied. For purposes of this invention, a “film’ is considered to be a thin (less than 5 mm thick) generally flexible sheet of polymeric material. Structured polymeric film layers can be micro-replicated. The provision of micro-replicated structured layers can provide surfaces that can be mass produced without substantial variation from product-to-product and without using relatively complicated processing techniques. “Microreplication” or “micro-replicated” means the production of a microstructured surface through a process where the structured surface features retain an individual feature fidelity during manufacture, from product-to-product, that varies no more than about 50 micrometers. The micro-replicated surfaces preferably are produced such that the structured surface features retain an individual feature fidelity during manufacture, from product-to-product, which varies no more than 25 micrometers. In accordance with some embodiments, a microstructured surface comprises a surface with a topography (the surface features of an object, place or region thereof) that has individual feature fidelity that is maintained with a resolution of between about 50 micrometers and 0.05 micrometers, more preferably between 25 micrometers and 1 micrometer. The channels of the fluid control films of the present invention can be any geometry that provides for desired liquid transport, and preferably one that is readily replicated. In some embodiments, the fluid control film will have primary channels on only one major surface. In other embodiments, however, the fluid control film will have primary channels on both major surfaces.

Certain of the fluid control films may be capable of spontaneously and uniformly transporting liquids (e.g., water, urine blood or other aqueous solutions) along the axis of the film channels. This capability is often referred to as wicking. Two general factors that influence the ability of fluid control films to spontaneously transport liquids are (i) the structure or topography of the surface (e.g., capillarity, shape of the channels) and (ii) the nature of the film surface (e.g., surface energy). To achieve the desired amount of fluid transport capability, the structure or topography of the fluid control film can be adjusted and/or the surface energy of the fluid control film surface can be adjusted. To achieve wicking for a fluid control film, the surface of the film should be capable of being “wet′ by the liquid to be transported.

Generally, the susceptibility of a solid surface to be wet by a liquid is characterized by the contact angle that the liquid makes with the solid surface after being deposited on a horizontally disposed surface and allowed to stabilize thereon. This angle is sometimes referred to as the ‘static equilibrium contact angle,” and sometimes referred to herein merely as “contact angle.” The contact angle Theta, θ, is the angle between a line tangent to the surface of a bead of liquid on a surface at its point of contact to the surface and the plane of the surface. A bead of liquid whose tangent was perpendicular to the plane of the surface would have a contact angle of 90°. If the contact angle is greater than 90°, the solid surface is considered not to be wet by the liquid and is referred to as being inherently “hydrophobic. Hydrophobic films include polyolefins, such as polyethylene or polypropylene. Typically, if the contact angle is 90° or less, the solid surface is wet by the liquid. Surfaces on which drops of water or aqueous solutions exhibit a contact angle of less than 90° are commonly referred to as “hydrophilic’.

As used herein, “hydrophilic’ is used only to refer to the surface characteristics of a material, i.e., that it is wet by aqueous solutions, and does not express whether the material absorbs aqueous solutions. Accordingly, a material may be referred to as hydrophilic whether a sheet of the material is impermeable or permeable to aqueous solutions. Thus, hydrophilic films used in fluid control films of the invention may be formed from films prepared from resin materials that are inherently hydrophilic, Such as for example, poly (vinyl alcohol). Liquids which yield a contact angle of near zero on a surface are considered to completely wet out the surface.

Depending on the nature of the micro-replicated film material itself, and the nature of the fluid being transported, one may desire to adjust or modify the surface of the film to ensure sufficient capillary forces of the film. For example, the structure of the surface of the fluid control film may be modified to affect the surface energy of the film. The fluid control films described herein may have a variety of topographies. As described above, preferred fluid control films are comprised of a plurality of channels with V-shaped or rectangular cross-sections, and combinations of these, as well as structures that have secondary channels, i.e., channels within channels. For open channels, the desired surface energy of the microstructured surface of V-channeled fluid control films is such that: Thetas (90°-Alpha2), wherein Theta (θ) is the contact angle of the liquid with the film and Alpha (C) is the average included angle of the secondary V-channel notches. It has been observed that secondary channels with narrower included angular widths generally provide greater vertical wicking distance. However, if Alpha is too narrow, the flow rate will become significantly lower. If Alpha is too wide, the secondary channel may fail to provide desired wicking action. As Alpha gets narrower, the contact angle Theta of the liquid need not be as low, to get similar liquid transport, as the contact angle Theta must be for channels with higher angular widths. Therefore, by modifying the geometry of the structured surface of the fluid control film, the surface energy and thus the wicking capability of the film may be modified to improve the liquid transport capability of the film.

Another example of modifying the surface of the film to ensure sufficient capillary forces of the film, is by modifying the surface to ensure it is sufficiently hydrophilic. Biological samples that will contact the fluid control films of the present invention are aqueous. Thus, if such films are used as fluid control films, they generally must be modified, e.g., by surface treatment, application of surface coatings or agents, or incorporation of selected agents, such that the surface is rendered hydrophilic so as to exhibit a contact angle of 90° or less, thereby enhancing the wetting and liquid transport properties of the fluid control film. Methods of making the surface hydrophilic include: (i) incorporation of a surfactant; (ii) incorporation or surface coating with a hydrophilic polymer; (iii) treatment with a hydrophilic silane; and (iv) treatment with an inorganic thin film coating such as SiO, which becomes hydrophilic upon exposure to moisture. Other methods are also envisioned. Other suitable known methods may achieve a hydrophilic surface on fluid control films. Surface treatments may be employed such as topical application of a surfactant, plasma treatment, vacuum deposition, polymerization of hydrophilic monomers, grafting hydrophilic moieties onto the film surface, corona or flame treatment, etc. An illustrative method for surface modification of the films of the present invention is the topical application of a one percent aqueous solution of a material comprising 90 weight percent or more of formula

Alternatively, a surfactant or other suitable agent may be blended with the resin as an internal additive at the time of film extrusion. Preferably, a surfactant can be incorporated in the polymeric composition from which the fluid control film is made rather than topical application of a surfactant coating because topically applied coatings tend to fill in, i.e., blunt, the notches of the channels, thereby interfering with the desired liquid flow to which the invention is directed. An illustrative example of a surfactant that can be incorporated in polyethylene fluid control films is TRITON™ X-100, an octylphenoxypolycthoxyethanol nonionic surfactant, e.g., used at between about 0.1 and 0.5 weight percent. Preferred embodiments retain the desired fluid transport properties throughout the life of the product into which the fluid control film is incorporated. To ensure the surfactant is available throughout the life of the fluid control film the surfactant preferably is available in sufficient quantity in the article throughout the life of the article or is immobilized at the surface of the fluid control film. For example, a hydroxyl functional surfactant can be immobilized to a fluid control film by functionalizing the surfactant with a di- or tri-alkoxy silane functional group. The surfactant could then be applied to the surface of the fluid control film or impregnated into the article with the article subsequently exposed to moisture. The moisture would result in hydrolysis and subsequent condensation to a polysiloxane. Hydroxy functional surfactants (especially 1.2 diol surfactants) may also be immobilized by association with borate ion. Suitable surfactants include anionic, cationic, and non-ionic surfactants; however, nonionic surfactants may be preferred due to their relatively low irritation potential. Polyethoxylated and polyglucoside surfactants are particularly preferred including polyethoxylated alkyl, aralkyl, and alkenyl alcohols, ethylene oxide and propylene oxide copolymers such as “Pluronic’ and “Tetronic’, alky lpolyglucosides, polyglyceryl esters, and the like.

Alternatively, a hydrophilic monomer may be added to the article and polymerized in situ to form an interpenetrating polymer network. For example, a hydrophilic acrylate and initiator could be added and polymerized by heat or actinic radiation. Suitable hydrophilic polymers include: homo and copolymers of ethylene oxide; hydrophilic polymers incorporating vinyl unsaturated monomers such as vinylpyrrolidone, carboxylic acid, sulfonic acid, or phosphonic acid functional acrylates such as acrylic acid, hydroxy functional acrylates such as hydroxyethylacrylate, vinyl acetate and its hydrolyzed derivatives (e.g. polyvinylalcohol), acrylamides, polyethoxylated acrylates, and the like: hydrophilic modified celluloses, as well as polysaccharides such as starch and modified Starches, dextran, and the like.

A hydrophilic silane or mixture of silanes may be applied to the surface of the fluid control film or impregnated into the article to adjust the properties of the fluid control film or article. Suitable silanes include the anionic silanes and non-ionic or cationic hydrophilic silanes. Cationic silanes may be preferred in certain situations and have the advantage that certain of these silanes are also believed to have antimicrobial properties.

Thin film inorganic coatings, such as SiO, may be selectively deposited on portions of the fluid control film or impregnated into the article, e.g., on the interior surface of microchannels. Deposition may occur either in-line during manufacture of the fluid control film or in a Subsequent operation. Examples of suitable deposition techniques include vacuum sputtering, electron beam deposition, solution deposition, and chemical vapor deposition. SiO coating of the fluid control film may provide the added benefit of producing a more transparent film than other types of coatings or additives. In addition, an SiO2 coating does not tend to wash off over time the way other coatings or additives may. The inorganic coatings may perform a variety of functions. For example, the coatings may be used to increase the hydrophilicity of the fluid control film or to improve high temperature properties. Application of certain coatings may facilitate wicking a sizing gel, filtration gel or assay reagent gel into the microchannels, for example. Conductive coatings may be used to form electrodes or diaphragms for piezoelectric or peristaltic pumping. Coatings may also be used as barrier films to prevent outgassing. An article, such as a wick, may be formed from a fluid control film having the capability of spontaneous fluid transport, as described above, and may be configured with either open or closed channels. For a closed channel wick made from a fluid control film to function, the wick is preferably sufficiently hydrophilic to allow the desired fluid to wet the surface of the fluid control film. For an open channel wick to function, the fluid must not only wet the surface of the fluid control film, but also the surface energy of the film must be at an appropriate level. Such that the contact angle Theta between the fluid and the surface is equal or less than 90 degrees minus one-half the notch angle Alpha, as set forth above.

FIG. 6 is a sectional view of the dressing 110 of FIG. 5 taken along line 6-6 of FIG. 5. The fluid control film 502 can have a first end 602 and a second end 604. The first end 602 of the fluid control film 502 can be disposed between the tissue interface 120 and the absorbent 210. For example, the first end 602 can be disposed in a fluid acquisition zone between the tissue interface 120 and the absorbent 210. The fluid acquisition zone can be an area of fluid absorption or fluid uptake by the absorbent 210. The second end 604 of the fluid control film 502 can be disposed in the timer 504. The second end 604 can be disposed in a fluid deposition zone. The fluid deposition zone can be an area of the timer 504 configured to receive fluid.

The fluid control film 502 can wick liquids from the fluid acquisition to the fluid deposition zone. As fluid is drawn by negative pressure through the tissue interface 120 into the absorbent, the fluid control film may wick the liquids from the fluid acquisition zone at a rate that is similar to the rate of the absorption of liquids by the absorbent 210. In some embodiments, as the liquid reaches the fluid deposition zone, the liquid travels at a rate that is clinically relevant to rates of exudate creation by the tissue site. As liquid is deposited in the fluid deposition zone, the timer 504 can transition from opaque to clear providing an indicator of a remaining amount of fluid absorption capacity of the absorbent 210 that is remaining.

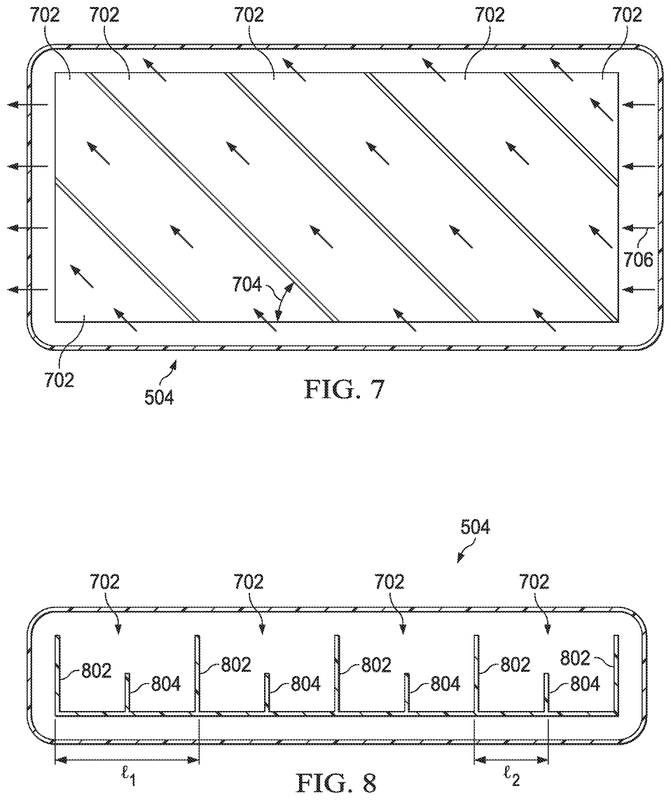

FIG. 7 is a plan view of the timer 504 of the dressing 110 of FIG. 5. The timer 504 may include a plurality of indicators 702 arranged parallel to each other and at an angle 704 to a flow of liquid 706. Each indicator 702 may receive liquid from the fluid control film 502 and separately transition from opaque to clear. The flow of liquid through the indicators 702 can correlate to a size-/diameter of the indicators 702 as well as the angle of fluid flow through the indicators 702. In some embodiments, the angle may be between about 30 degrees and about 60 degrees and preferably 45 degrees. Preferably, the indicators 702 in closest proximity to the flow of liquid 706 may transition from opaque to clear first. As more liquid reaches the timer 504 more indicators 702 may transition, giving a user a visual representation of a saturation state of the absorbent 210. In some embodiments, if the indicators 702 are clear, the indicators 702 may have a transparency of between 50% to 90% or may increase in transparency by at least 25%.

FIG. 8 is a front view of the timer 504 of FIG. 7. In some embodiments, the indicators 702 can comprise a plurality of channels having walls 802 and walls 804. In some embodiments, a height of the walls 804 may be less than a height of the walls 802. The walls 802 may be spaced from each other a distance 11. The walls 804 may be disposed between adjacent walls 802 so that a wall 804 separates adjacent walls 802. Similarly, a wall 802 may be disposed between adjacent walls 804. In some embodiments, the wall 804 may be spaced from a wall 804 by a distance 12. In some embodiments, the distance 12 may be one-half the distance 11. The distances 11 and 12 can be varied to control the rate of flow of capillary action under negative pressure. For example, the distances 11 and 12 can be increased to slow the rate of capillary action, causing the indicators to transition from opaque to clear at a slower rate. Conversely, the distances 11 and 12 can be decreased to increase the rate of capillary action, causing the indicators to transition from opaque to clear at a quicker rate.

As disclosed herein, the therapy system 100 can provide a visual indication of when it is appropriate to remove an absorbent dressing. Furthermore, the therapy system 100 can prevent the early unnecessary removal of a dressing. By providing visual indications of the saturation status of the absorbent within the dressing, the risk of maceration and contamination of the tissue site is reduced.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing for treating a tissue site, the dressing comprising:
a tissue interface having a fluid flow path;
an encapsulating film at least partially encapsulating the tissue interface; and
at least one indicator disposed adjacent to the tissue interface proximate to at least one location along the fluid flow path.

2. The dressing of claim 1, wherein:
the fluid flow path comprises a serpentine flow path having at least one bend; and
the at least one location comprises the at least one bend.

3. The dressing of claim 1, wherein the indicator comprises a film defining a portion of the fluid flow path, the film having an ultra-low index coating.

4. The dressing of claim 3, wherein the ultra-low index coating is configured to transition between opacity and transparency in a presence of liquid.

5. The dressing of claim 4, wherein the ultra-low index coating is configured to transition from opaque to transparent in the presence of liquid.

6. The dressing of claim 1, wherein the indicator comprises:
a plurality of microfluidic channels configured to draw fluid into each microfluidic channel of the plurality of microfluidic channels; and
the at least one location comprises a first end of each channel of the plurality of microfluidic channels disposed at an interface between the tissue interface and the tissue site and a second end of each channel of the plurality of microfluidic channels disposed between the tissue interface and the encapsulating film.

7. The dressing of claim 6, wherein the plurality of microfluidic channels are disposed at an angle to the fluid flow path.

8. A system for treating a tissue site, the system comprising:
a tissue interface having a fluid flow path;
a pouch at least partially encapsulating the tissue interface;
at least one saturation meter disposed adjacent to the tissue interface proximate to at least one location along the fluid flow path; and
a negative-pressure source configured to be fluidly coupled to the tissue interface to draw fluid along the fluid flow path.

9. The system of claim 8, wherein:
the fluid flow path comprises a serpentine flow path having at least one bend; and
the at least one location comprises the at least one bend.

10. The system of claim 8, wherein the saturation meter comprises a film defining a portion of the fluid flow path, the film having an ultra-low index coating.

11. The system of claim 10, wherein the ultra-low index coating is configured to transition between opacity and transparency in a presence of liquid.

12. The system of claim 11, wherein the ultra-low index coating is configured to transition from opaque to transparent in the presence of liquid.

13. The system of claim 8, wherein the saturation meter comprises:
a plurality of microfluidic channels configured to draw fluid into each microfluidic channel of the plurality of microfluidic channels; and
the at least one location comprises a first end of each channel of the plurality of microfluidic channels disposed at an interface between the tissue interface and the tissue site and a second end of each channel of the plurality of microfluidic channels disposed between the tissue interface and the encapsulating film.

14. The system of claim 13, wherein the plurality of microfluidic channels are disposed at an angle to the fluid flow path.

15. The system of claim 8, further comprising a manifold disposed between the tissue interface and the tissue site, the manifold configured to direct fluid flow from the tissue site to the tissue interface.

16. A method of manufacturing a dressing for treating a tissue site, the method comprising:
providing a tissue interface having a fluid flow path;
at least partially encapsulating the tissue interface in a film; and
disposing at least one indicator adjacent to the tissue interface proximate to at least one location along the fluid flow path.

17. The method of claim 16, wherein the method further comprises:
forming at least one bend in the fluid flow path; and
disposing the at least one indicator comprises disposing the at least one indicator at the at least one bend.

18. The method of claim 16, wherein the method further comprises:
coating the film defining a portion of the fluid flow path with an ultra-low index coating to dispose the indicator.

19. The method of claim 16, wherein the disposing the at least one indicator comprises:
forming a plurality of microfluidic channels in the encapsulating film, the plurality of microfluidic channels configured to draw fluid into each microfluidic channel of the plurality of microfluidic channels; and
disposing a first end of each channel of the plurality of microfluidic channels at an interface between the tissue interface and the tissue site and a second end of each channel of the plurality of microfluidic channels between the tissue interface and the encapsulating film.

20. The method of claim 19, wherein the method further comprises disposing the plurality of microfluidic channels at an angle to the fluid flow path.

21. (canceled)
